(19) United States
(12) Patent Application Publication
(10) Pub. No.: US 2020/0179632 A1
(43) Pub. Date: Jun. 11, 2020

(54) INTRAORAL DEVICE FOR GAS DELIVERY AND MONITORING

(71) Applicant: Gregory Worley, Pompano Beach, FL (US)

(72) Inventor: Gregory Worley, Pompano Beach, FL (US)

(21) Appl. No.: 16/214,707

(22) Filed: Dec. 10, 2018

(51) Int. Cl. A61M 16/04; A61M 16/06; A61M 16/08

(52) U.S. Cl. CPC A61M 16/0493; A61M 16/0666; A61M 16/085; A61M 2230/432; A61M 16/0495; A61M 2202/0208; A61M 16/042

(57) ABSTRACT

Described is device designed to maintain an open airway for use in an anesthetized patient undergoing a medical procedure or surgery while affording oropharyngeal insufflation and expiratory monitoring of the patient. The device is fashioned as an intraoral bite block having one or two pre-formed conduits for insufflation or exhalation monitoring which are readily coupled to a conventional nasal cannula. Also described are a method of using the device, and a system for providing oral insufflation to an anesthetized patient using a bite block as described in combination with a conventional nasal cannula.

### FIELD OF INVENTION

The present invention relates to medical devices and methods useful in an anesthetized patient undergoing a surgical or medical procedure of the head, neck or face. More particularly, the present invention is directed to an intraoral bite block or oropharyngeal airway device for performing, or facilitating the performance of, patient insufflation and CO₂ monitoring.

### BACKGROUND OF THE INVENTION

It is well known in the field of human anesthesia to employ a nasal cannula for delivering oxygen (O₂) to an anesthetized patient and to monitor end-tidal carbon dioxide (ETCO₂) from the patient's exhaled air. Typically, a supply (insufflation) line extends from a gas supply source and connects with an inlet of the cannula to supply oxygen or other treatment gas to the patient while an exhalation monitoring tube connects to an outlet of the cannula to return expired gas to a monitoring/capnography system. Alternatively, both sides may be utilized for insufflation or for directing expired gas to a monitoring source.

Most conventional nasal insufflation and monitoring cannulae comprise two ports, each port configured to correspond to one nare of a patient, as shown for example in U.S. Pat. No. 8,683,998 to Swanson. The ports are typically in respective communication with a gas delivery tube or line connected to an oxygen source, and an exhalation tube or line connected to a CO₂ monitoring device for sampling and measuring the patient's ETCO₂. Additional embodiments of such cannulae known in the art and are described, for example, in U.S. Pat. Nos. 6,938,619; 4,989,599; 6,439,234; and 5,046,491. None of these nasal cannulae are designed for intraoral use, however.

Oxygen insufflation and ETCO₂ monitoring through nasal cannulae may be suboptimal in certain instances, for example, in cases where the nasal passages are blocked or during certain procedures such as cosmetic or facial reconstructive surgery where the physician may require unencumbered access to the nasal area of the patient.

In such situations, the physician may opt for insufflation and monitoring via an intraoral route rather than nasal route. When using an intraoral route, the physician or anesthetist typically directs an insufflation tube and a respiration monitoring tube into the patient's mouth for intraoral and pharyngeal access. Devices known as modified bite blocks are often employed for this use as they serve to hold the patients mouth open and provide an access path for surgical instruments or for endoscopic or intubation devices as well as gas insufflation and exhalation monitoring lines. Such modified bite blocks are well known in the medical and dental arts and are available in a variety of configurations as exemplified in U.S. Pat. No. 6,098,617.

In certain medical or surgical procedures such as in facial reconstruction and cosmetic surgery however, there may be no need for an airway device such as conventional intraoral bite blocks to accommodate the insertion of surgical instruments, endoscopic devices or intubation apparatus, but gas insufflation and exhalation monitoring is still required for proper anesthesia protocol. Accordingly, standard airway bite blocks having such an accommodation are often larger and more cumbersome than necessary, especially when performing procedures around the mouth and under the nose.

Accordingly, it would be desirable to have an improved method and device for providing oral insufflation and exhalation monitoring to a patient where nasal cannulation is not practical and there is no need to accommodate insertion of surgical instruments, endoscopic devices or intubation apparatus.

Additionally, it is frequently the case that a patient undergoes a surgical procedure involving both the nose and the mouth as frequently occurs with various types of facial cosmetic surgery. In such situations, readily switching between nasal insufflation and intraoral insufflation at desired times is preferred. Such a switch is often cumbersome and time consuming using conventional equipment and methods. Accordingly, it would be advantageous to have a method and device for rapidly switching between the two routes.

### SUMMARY OF THE INVENTION

The present invention is directed to an intraoral bite block for respiratory insufflation and exhalation monitoring of an anesthetized patient where there is no need for intubation or insertion of surgical instruments or endoscopes through the bite block. The bite block has a proximal facial end for external placement outside the patient's oral cavity and a distal pharyngeal end for insertion into the patient's oral cavity and pharyngeal area. The proximal facial end includes an external lip flange for placement outside the oral cavity where it rests against and engages the external aspect of the patient's lip or lips to secure and prevent aspiration of the device.

More particularly, the bite block of the present invention comprises an elongate body having a proximal or facial end for external placement outside the patient's oral cavity and a distal or pharyngeal end for insertion into the patient's oral cavity and pharyngeal area (the oropharyngeal area), the ends opposite one another relative to the longitudinal axis of the elongate body.

The elongate body also includes a bite surface section comprised of an upper and lower bite surface positioned adjacent and distal to the lip flange to mate with the upper and lower dental arches of the patient, respectively. The bite surface section preferably serves to matingly engage the occlusal surface of the patient's maxillary (upper) and mandibular (lower) dental arches or teeth, thereby preventing dental occlusion and consequent damage or occlusion of a conduit carrying the gas to and from the patient.

The elongate body should have sufficient length to extend distally into the oropharyngeal cavity of the patient. The pharyngeal section is preferably curved, being configured to approximate the conformation of the patient's oral and pharyngeal anatomy. The elongate body thereby serves to depress the tongue and pharyngeal tissues for creation and maintenance of an open airway during use.

The bite block further includes at least one, but no more than two, delivery conduits disposed along its longitudinal axis from the distal pharyngeal end to the proximal facial end where each conduit terminates in a conduit matingly connectable with and rapidly detachable from a corresponding port of a nasal cannula. The delivery conduits can alternatively be provided having a length that extends to the gas delivery source and exhalation monitor (for measurement and monitoring of carbon dioxide, or CO₂ levels.)

The bite block of the present invention can be advantageous for certain treatments and surgical procedures where gas insufflation and exhalation monitoring only by the nasal route is not practical or where there is no need to accommodate insertion of surgical instruments, endoscopic devices or intubation apparatus.

Because the inventive bite block is designed to address only gas insufflation and monitoring, the device excludes an additional conduit to accommodate endoscopic tools and the like. This affords a smaller, less obstructive bite block advantageous over other larger bite blocks having three or more conduits that may compromise surgical procedures involving the area around the mouth.

Additionally, since the bite block of the present invention is relatively small in circumference or radial size, it requires minimal oral clearance, and can provide for more relaxation of the facial muscles during facial surgery. This aids the physician in performing certain contouring and re-structuring procedures and promotes a better aesthetic result.

Thus, as described herein and illustrated in the accompanying drawings, the subject invention is an oropharyngeal airway device or intraoral bite block for preventing dental occlusion and providing for oral insufflation of an anesthetized patient using a nasal cannula, wherein the bite block comprises:
an elongate body having a proximal and a distal end, the ends opposite one another relative to the longitudinal axis of the elongate body, wherein the proximal end terminates in at least one lip flange which engages an external aspect of a lip of the patient, the elongate body further comprising an upper and lower bite surface positioned adjacent and distal to the lip flange to mate with the upper and lower dental arches of the patient, respectively, the elongate body having a length sufficient to extend distally into the oropharyngeal cavity of the patient, the elongate body further comprising at least one, but not more than two, gas conduits extending distally along the longitudinal axis of the elongate body from the lip flange and extending distally beyond the upper and lower bite surface with each conduit being configured at its proximal end to, in a preferred embodiment, matingly and detachably couple with a port of a nasal cannula. The bite block can comprise a conduit formed along the longitudinal axis of the elongate body wherein the conduit is sized to receive an oxygen insufflation tube and an exhalation tube. The conduit can be divided by a partitioning septum forming first and second conduits, each conduit sized to receive an insufflation tube or an exhalation tube.

The septum is preferably formed in a vertical orientation along the longitudinal midplane of the elongate body, the septum forming a right and a left channel for disposing and affixing an insufflation tube in one of said right or left channels and disposing and affixing an exhalation tube in the other channel. The insufflation tube can transport oxygen to the patient and the exhalation tube can transport exhaled gas from the patient. The insufflation tube and exhalation tube can be permanently affixed within the channels along the longitudinal axis of the elongate body.

The lip flange can be integrally formed with the elongate body of the bite block or can be formed separately and affixed to the elongate body during assembly or manufacture of the bite block.

The bite surface can comprise a groove to receive occlusal surfaces of teeth of the patient and can comprise a cushioned outer surface, either being formed from a cushioning material or having a cushioning material covering the bite surface.

The bite block preferably comprises two lip flanges, a first lip flange being an upper lip flange extending superiorly and perpendicular to the longitudinal axis of the elongate body and a second lip flange being a lower lip flange extending inferiorly and perpendicular to the longitudinal axis of the elongate body.

The elongate body portion of the bite block of the subject invention preferably serves as a tongue depressor for maintaining an open oropharyngeal airway in an anesthetized patient. In addition, the elongate body can be substantially curvature in shape, being curved for anatomical conformity with the oropharyngeal cavity of the patient.

In one preferred embodiment of the invention, the elongate body of the bite block has a cross-sectional “I” shape wherein a longitudinal partitioning septum is disposed in a vertical orientation along the longitudinal midplane of the elongate body. Substantially planar upper and lower projections are formed perpendicular to the septum thereby forming two channels on each side of the septum, wherein a first conduit for gas insufflation is disposed and affixed within one channel and a second conduit for gas exhalation is disposed and affixed within the other channel.

The channels formed in this embodiment of the invention can comprise open sides or can comprise exterior side walls parallel to the middle vertical septum forming closed channels.

The gas conduit can connect directly to a corresponding cannula port or can comprise a connector disposed between the proximate end of the conduit and the port of the nasal cannula. The connector is preferably permanently affixed to the conduit. In either embodiment, the conduit or connector preferably extends out past the lip flange to facilitate coupling of the bite block and the cannula. In an embodiment, the conduit or connector can be flush with, or recessed within an outer surface of the lip flange.

In a preferred embodiment of the bite block of the subject invention, the inhalation conduit and an exhalation conduit are spatially configured to matingly couple to inhalation and exhalation ports of a conventional nasal cannula.

Advantageously, the bite block of the subject invention excludes a third conduit for access through the bite block by medical or surgical instruments, endoscopes, or intubation tubes. The absence of a third conduit results in a smaller bite block, which can advantageously be more comfortable for the patient, both during and after the procedure. A smaller bite block can also be advantageous in providing a more natural position of the jaw and face for facial reconstructive surgery.

Use of the bite block of the invention includes a method for providing oral insufflation to, and exhalation monitoring of, an anesthetized patient undergoing a procedure requiring access to an area of the face or head, the method comprising the steps of (a) positioning the intraoral bite block into an oropharyngeal cavity of the anesthetized patient, and (b) detachably coupling a nasal cannula to the bite block to avoid cannular interference with access to the face or head area undergoing the procedure. The intraoral bite block can advantageously remain in the oropharyngeal cavity when the nasal cannula is detached from the bite block and positioned for nasal insufflation.

The invention further comprises a system for alternatively providing oral and nasal gas insufflation to, and exhalation monitoring of, an anesthetized patient, the system comprising wherein the system includes the components and use of an intraoral bite block comprising an elongate body, at least one lip flange and at least one, but not more than two, gas conduits matingly and detachably coupling with a port of a nasal cannula, as described, and a nasal cannula for providing gas insufflation and exhalation monitoring of a patient, the cannula being capable of matingly coupling with and rapidly detachable from said bite block.

In a preferred embodiment, the system of the invention can include a cannula comprising insufflation tubing extendable to and connectable with an insufflation gas source and exhalation tubing extendable to and connectable with a gas monitor, such as capnography equipment, wherein the insufflation tubing and exhalation tubing can be drawn together and adjoined in a side-by-side configuration by an adjoining means engaging each of the insufflation tubing and exhalation tubing, the adjoining means being slidingly adjustable along the lengths of said tubing.

### BRIEF DESCRIPTION OF THE DRAWINGS

The drawings as provided for illustrating exemplary embodiments of the present invention, the detailed description of which follows hereinbelow. The drawings are not intended to limit the invention as encompassed by the claims appended herewith.

FIG. 1 is a lateral perspective view of the bite block of the present invention.

FIG. 2 is a top elevation view of the bite block of the present invention in communication with a nasal cannula.

FIG. 3 is a lateral view of the bite block of the present invention in communication with a nasal cannula.

FIGS. 4 and 5 show lateral perspective views of both sides of the bite block of the present invention in communication with a nasal cannula.

FIG. 6 is a frontal view of the bite block of he present invention in communication with a nasal cannula.

FIG. 7 is a view of a patient with nasal placement of a dual nasal cannula as contemplated for use with the system and methods of the present invention

FIG. 8 is a view of the bite block of the present invention disposed within the oral cavity of a patient, the bite block in communication with a nasal cannula having inbound and outbound lines that feed into the cannula from a substantially horizontal direction perpendicular to the medial plane of the patient.

FIG. 9 is a view of the bite block of the present invention disposed within the oral cavity of a patient, the bite block in communication with a nasal cannula having inbound and outbound lines that feed into the cannula from a substantially vertical direction parallel with the medial plane of the patient.

### DETAILED DESCRIPTION OF THE INVENTION

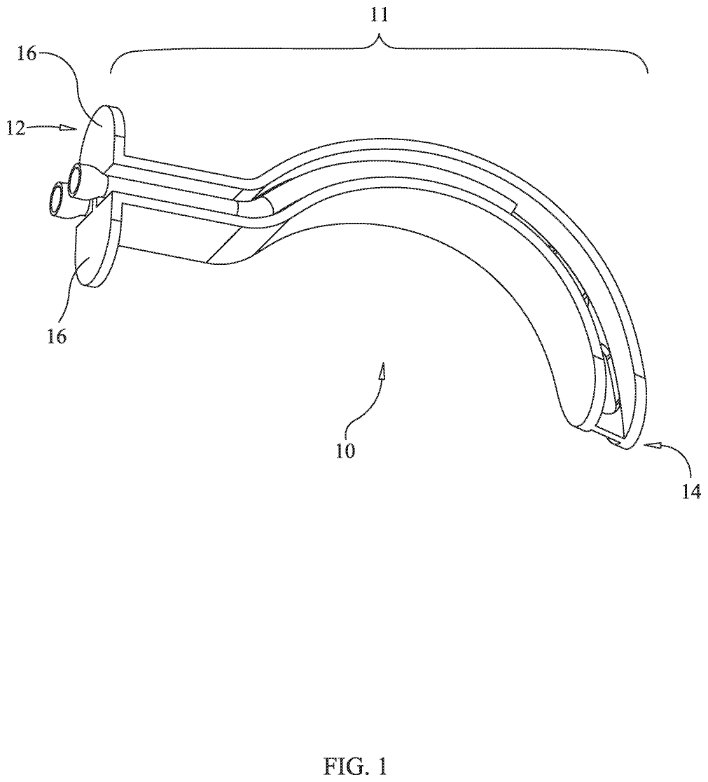

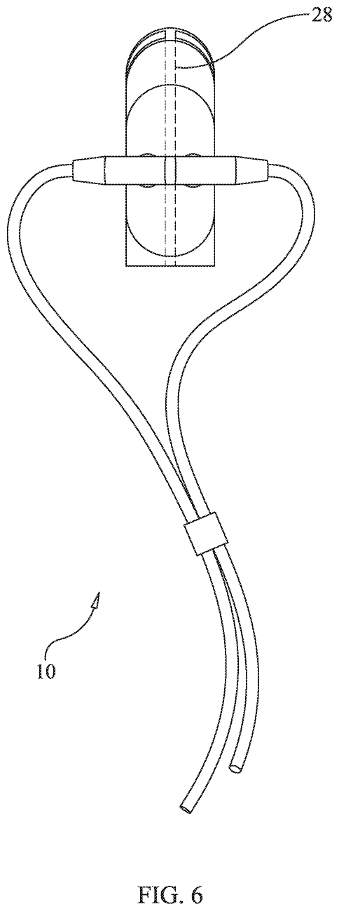

The following description is made in general reference to FIGS. 1-9 and is provided herewith to illustrate exemplary embodiments of the present invention.

In accordance with the present invention, there is described herein an intraoral bite block for respiratory insufflation and exhalation monitoring of a patient Referring to the figures, preferred embodiments of the subject device are shown.

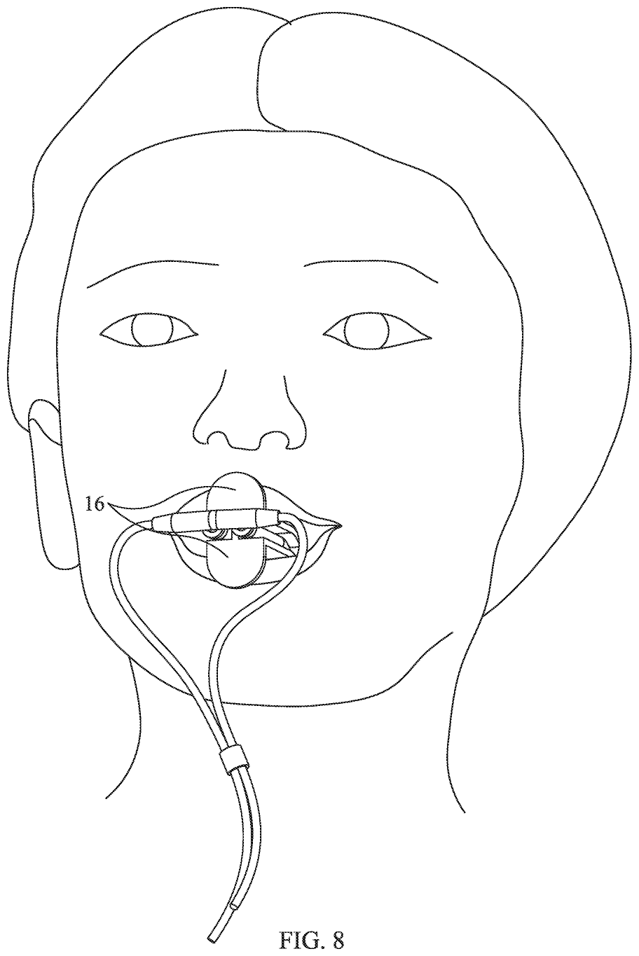

Turning to FIG. 1, the intraoral bite block 10 comprises elongate body 11 having a proximal or facial end 12 for external placement outside the patient's oral cavity and a distal or pharyngeal end 14 for insertion into the patient's oral cavity and pharyngeal area (the oropharyngeal area), the ends opposite one another relative to the longitudinal axis of the elongate body. Proximal or facial end 12 is preferably fashioned as one or more external lip flanges 16 for placement outside the oral cavity where the flange rests against the external aspect of the patient's lips to secure and prevent aspiration of the device as shown for example in FIG. 8.

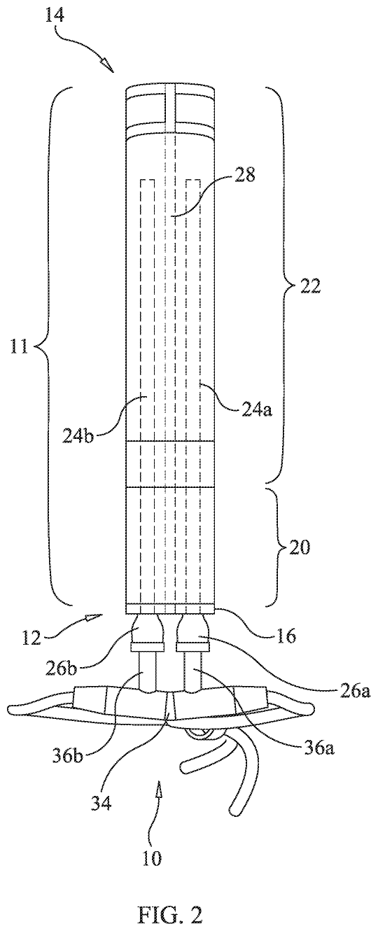

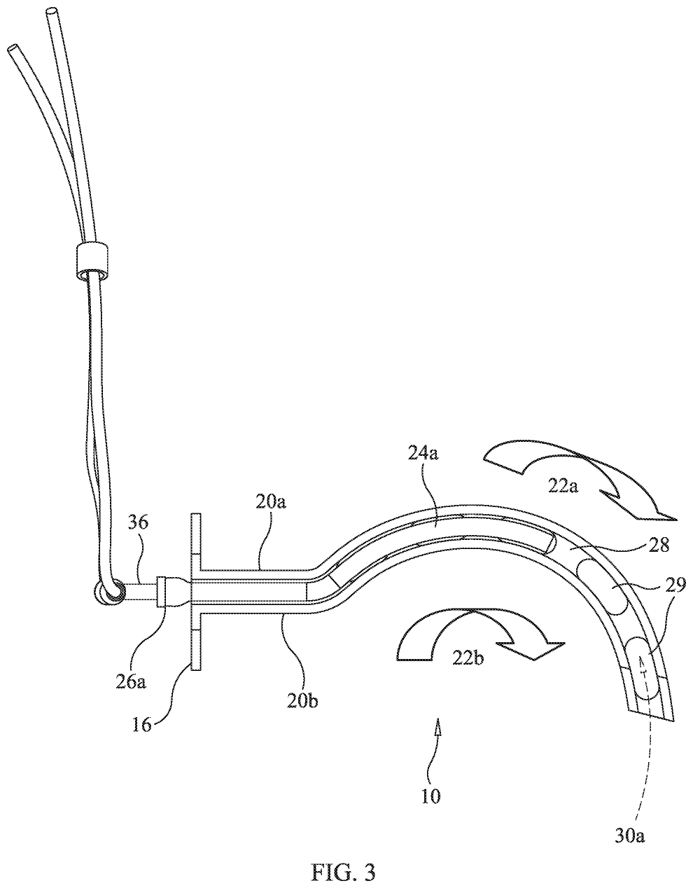

Turning to FIGS. 2 and 3, the elongate body 11 preferably includes a bite surface section 20 and pharyngeal section 22 that is preferably configured to approximate the general curvature of the patient's oral and pharyngeal anatomy to sufficiently depress the tongue and pharyngeal tissues for creation and maintenance of an open airway during use. It will be appreciated by the artisan that bite surface 20 should preferably comprise an upper and lower bite surface positioned adjacent and distal to the lip flange to mate with the upper (maxillary) and lower (mandibular) dental arches of the patient, respectively, while the elongate body should have a length sufficient to extend distally into the oropharyngeal cavity of the patient by way of pharyngeal section 22.

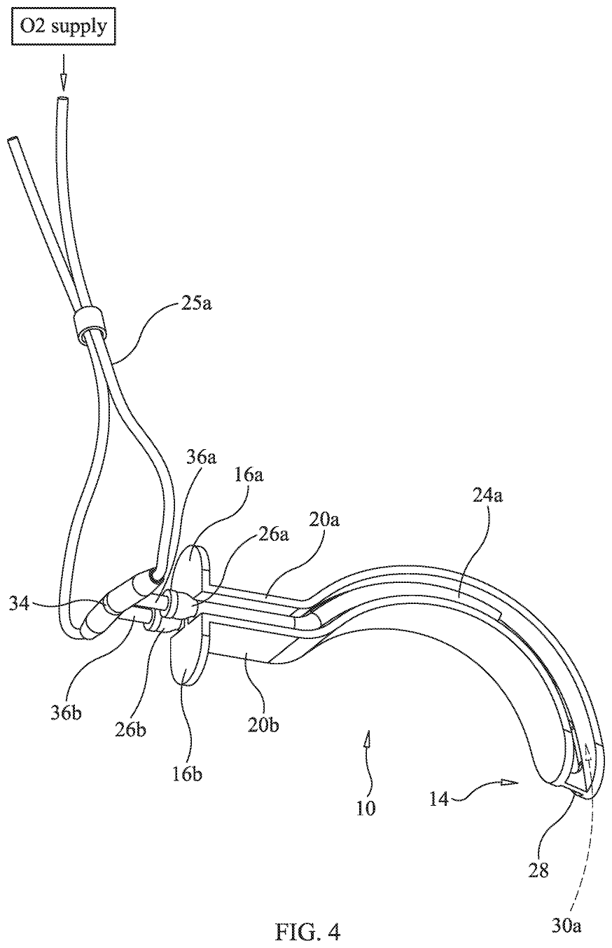

Elongate body 11 further includes at least one, but not more than two, gas conduits disposed along the longitudinal axis of the elongate body from the lip flange and extending distally beyond the upper and lower bite surface. For example, the drawings show one typical embodiment of the invention having two (first and second) conduits, 24a and 24b, both independently disposed along the longitudinal axis of the elongate body from proximal end 12 (where each conduit terminates in connectors 26a and 26b, respectively) and extending distally beyond bite surface section 20, as shown in FIGS. 3 and 4.

It is important to note that the connectors 26a and 26b are shown as extending out, beyond the outer (proximal) surface of the lip flange. This is for illustration purposes only. It would be understood that the connectors can end flush with the outer surface of the lip flange or can be recessed relative to the outer surface of the lip flange. The position relative to the outer surface of the lip flange is relevant only to the extent that the nasal cannula can be detachably affixed to the device for its operation during use. It would also be understood that the nasal cannula connection with the conduit can be served by the configuration of the conduit being such that it can receive and connect to the nasal cannula ports without separate connector elements.

In one alternative embodiment, channels can comprise outer walls along the sides of the device which can facilitate guidance or placement of the gas conduits. The front or proximal ends of the channels can be configured having apertures having a diameter for snugly receiving the gas conduits. Alternatively, the gas conduits can be formed, per se, by enclosed channels, having outer walls disposed along the longitudinal axis of the elongate body portion of the device, and thereby eliminating any requirement for tubing or additional conduit means to deliver oxygen to, and transport exhaled gases (e.g., CO₂) from the patient.

It would be readily understood that separate connectors are optional and that the conduit ends or the proximal ends of the enclosed channels can be configured to matingly couple and detachably connect with the nasal cannula ports directly.

Turning to FIG. 2, elongate body portion 11 is shown to include bite surface section 20 and a pharyngeal section 22. Bite surface section 20 preferably includes maxillary bite surface 20a fashioned to engage at least a portion of the maxillary arch of the patient and a mandibular bite surface 20b fashioned to engage at least a portion of the mandibular arch of the patient, both of which are best seen in FIG. 3. It will be appreciated that while bite surface 20 is shown in the Figures as a planar biting surface, those skilled in the art will recognize that it may also be fashioned as a groove, an indentation or other receiving surface suitable for receiving the maxillary and mandibular arches of a patient, whether or not edentulous. The bite surface can include a cushioned surface or cushioned cover for comfort of the patient.

Elongate body portion 11 includes at least one lip flange which extends over the outer surface of the upper and lower lip of the patient. This flange can be fashioned as a unitary flange, integrally formed with the elongate body of the bite block, or the lip flange can be fashioned as a separate piece which is affixed to and becomes contiguous with the elongate body during manufacture, as long as it adequately serves to secure the bite block and prevent movement of the block during use and protects against possible aspiration of the block. In one preferred embodiment shown in FIG. 4, for example, lip flange 16 further comprises a maxillary flange 16a which connects to and extends from maxillary surface 20a to engage the external aspect of the upper lip of the patient and mandibular flange 16b which connects to and extends from mandibular surface 20b to engage the external aspect of the lower lip of the patient.

As described and shown, the pharyngeal section 22 of elongate body 11 is preferably configured to approximate the general curvature of the patient's oral and pharyngeal anatomy to sufficiently depress the tongue and pharyngeal tissues for creation and maintenance of an open airway during use. As shown in FIG. 3, pharyngeal section 22 is preferably formed having over curve 22a and under curve 22b wherein the top aspect of pharyngeal section 22 contacts the upper ridge of the patient's mouth at the hard palate just before the soft palate and wherein the bottom aspect of pharyngeal section 22 functions as a tongue depressor to maintain the tongue in a posterior and downward position to facilitate an open airway. It will be appreciated by those skilled in the art that the pharyngeal section should be of sufficient length to maintain an open airway yet avoid triggering any gag reflex by the patient when inserted and correctly positioned.

In one preferred embodiment of the invention, elongate body 11 preferably comprises a longitudinal partitioning septum 28 disposed in a vertical orientation along the longitudinal midplane of the elongate body, the septum forming at least two channels, 30a and 30b, disposed along the elongate body longitudinal axis with each channel being discretely disposed on one side or the other of partitioning septum 28 as shown in FIGS. 3-5. This arrangement provides a right and a left channel for disposing and affixing an insufflation tube in one of the channels and disposing and affixing an exhalation tube in the other channel as shown in FIGS. 2-4 and 6.

Partitioning septum 28 may be formed as a solid wall or as a wall with optional apertures 29 if desired. It will be appreciated by those skilled in the medical device manufacturing arts that while partitioning septum 28 may be independently fabricated and subsequently affixed within bite block of the present invention by any of numerous attachment means available to the artisan, the septum is preferably formed integrally with the elongate body, and preferably disposed along the longitudinal midline of body 11 at the time of its manufacture to form at least one channel disposed along the longitudinal axis of body 11. Typically, the elongate body will have a cross-sectional “I” shape with the longitudinal partitioning septum disposed in a vertical orientation along the longitudinal midplane of the elongate body with substantially planar upper and lower projections formed perpendicular to the septum in order to form two channels, each one side positioned on the opposite side of the partitioning septum with respect to the other.

By way of example only, the channels can be circular in shape, and sized to snugly receive tubing as described below. Alternatively, the channels may be formed to approximate an ovoid, rectangular, or other geometric shape, wherein tubing can be affixed or adhered to the inner wall of the channel.

In the event that the intraoral bite block of the present invention is formed with channels, 30a and 30b, it will be appreciated that gas delivery conduits 24a and 24b will preferably be disposed within the channels and affixed therein either by way of a suitable attachment means such as a medical grade glue or secured by way of dimensional fit. Accordingly, depending upon the width of the channel, an appropriately sized conduit having dimensions easily ascertained by those skilled in the medical device engineering and fabrication arts can be seated within the channel and secured therein by virtue of size. If such a design is employed, gas delivery conduits 24a and 24b can be formed from properly sized medical grade respiratory tubing readily known and commercially available to the artisan.

It will be further appreciated by the artisan that delivery conduits 24a and 24b may not be required to run the entire longitudinal axis of elongate body 11 so long as they reach an adequate depth within the patient's anatomical airway to properly deliver an insufflation gas and/or to properly acquire a sufficient sampling of the patient's exhaled air for transport and monitoring, preferably distal to bite surface 20. Accordingly, partitioning septum 28 may similarly not run the entire longitudinal axis of elongate body 11 so long as it provides an adequate channel for seating and securing the delivery conduits.

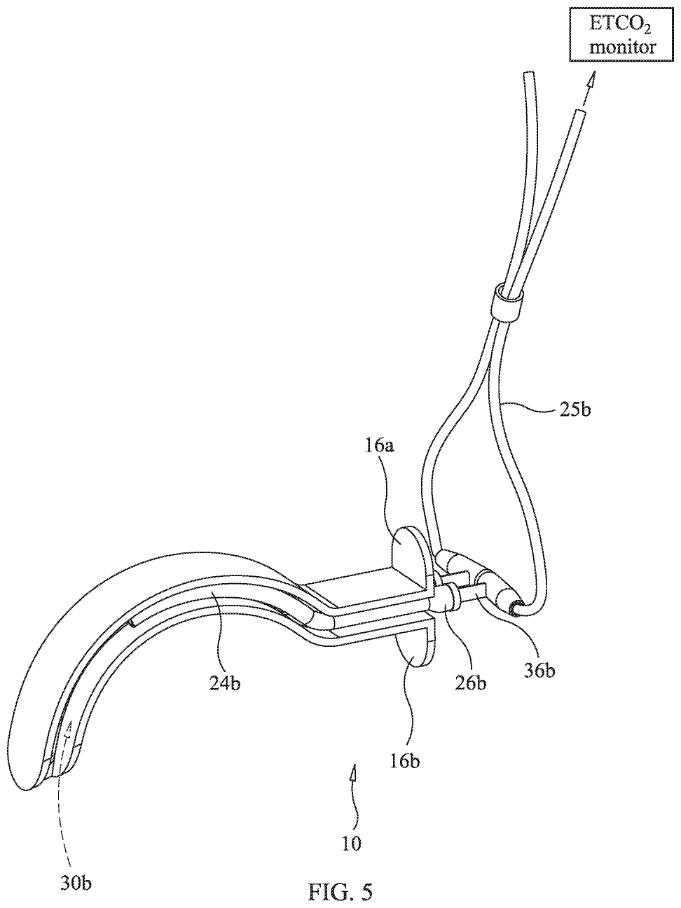

In the event that the bite block is fabricated to include two channels, as exemplified by 30a and 30b, it will be preferable for one of the delivery conduits to carry an insufflation gas while the remaining conduit is utilized for sampling the patient's exhaled air for monitoring. Typically, the first conduit will provide inbound delivery of oxygen to the patient by way of inbound line 25a shown in FIG. 4 while the second delivery conduit will provide transport of outbound exhaled air to outbound line 25b to allow end-tidal carbon dioxide sampling as shown in FIG. 5.

Each delivery conduit of the inventive bite block terminates at the proximal facial end of the bite block in a conduit that is matingly and manually connectable with and rapidly detachable from one port of a nasal cannula with or without a separate adaptor. Alternatively, the bite block can comprise connectors or fittings formed integrally with or separately affixed to the conduit to receive and engage the cannular ports. For example, turning to FIGS. 2-5, delivery conduits 24a and 24b terminate at proximal end 12 of elongate body 11 with connectors 26a and 26b, respectively, which in turn matingly engage cannular ports 36a and 36b.

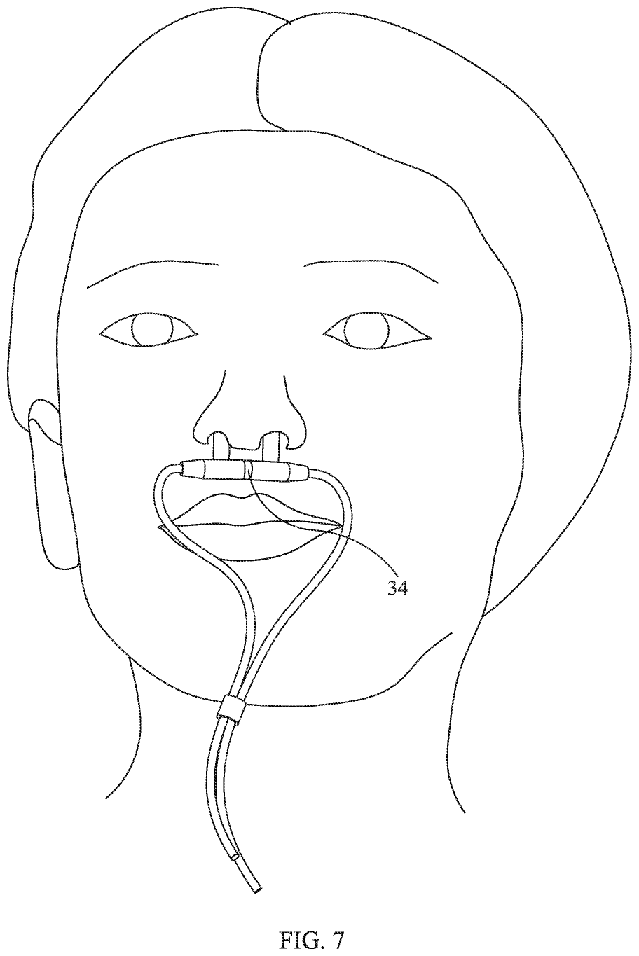

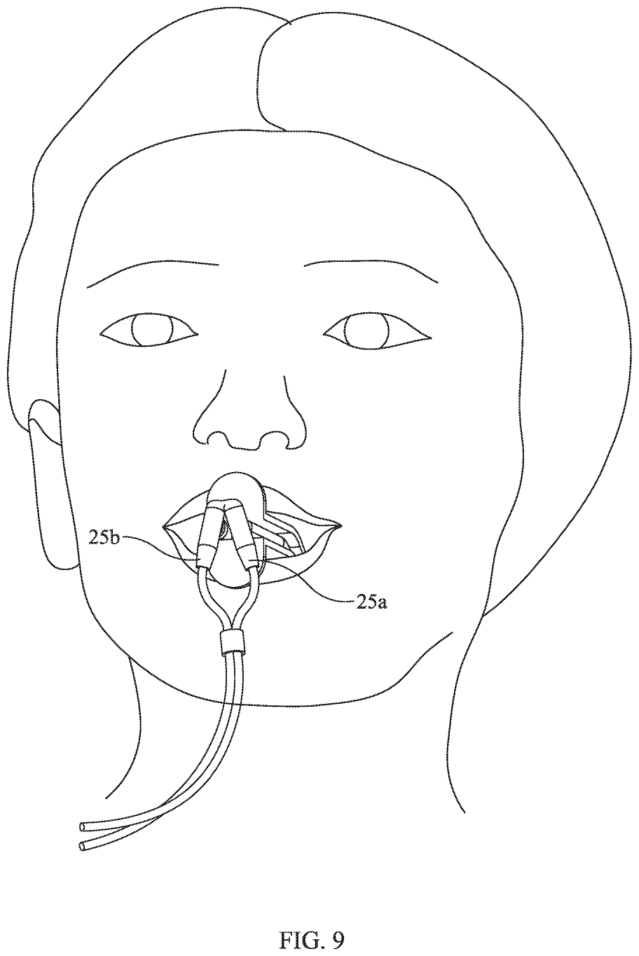

Additionally, it will be further appreciated by those skilled in the art that the intraoral bite block may be utilized in a system comprising a bite block, as described herein, and a dual-purpose cannula suitable for use as a conventional nasal cannula wherein the cannula can, in addition to functioning at the nasal passage of a patient to perform insufflation and monitoring as shown in FIG. 7, similarly perform insufflation and monitoring by engaging the bite block of the present invention to provide oral delivery of an insufflation gas to the patient as well as oral monitoring of exhaled gas as shown in FIGS. 8 and 9.

As described above, the connectivity between the cannular ports and the conduits of the bite block of the invention may also include any of various adapters known in the respiratory arts to further facilitate the rapid engagement of the cannula for oral insufflation gas delivery and exhalation monitoring by way of the bite block. Such adapters can be deployed as discrete intermediary connections, or alternatively formed as part of either the cannular ports or delivery conduits of the bite block.

The dual-purpose cannula of the system is preferably adapted to allow for up to a 90° bend in the cannula typically along the cannular midline, at cannula partition 34, as best illustrated in FIGS. 2, 4 and 7 in conjunction with FIG. 9. Typically, the cannula midline 34 is a partition formed between the delivery side (for delivery of oxygen) of the nasal cannula and the expiration side (for receiving and monitoring exhalation gases) of the cannula. In a preferred embodiment, the cannula partition is partially split or divided to allow each partition side to bend approximately 90° and parallel to one another (see the cannula in bent configuration in FIG. 9). In the prototypes of the invention, a standard, commercially available cannula was modified by partially splitting the partition along the midline (not completely through to separate the two sides, but at a distance through the midline which allows for the desired bending without complete separation of the two sides of the nasal cannula. It would be understood that a cannula having a partially divided septum can be formed at the time of manufacture of the cannula. This adaptability advantageously allows the cannula to assume a varied configuration ranging from a position where the cannula is aligned in a substantially horizontal orientation across the labial axis as shown in FIGS. 7 and 8 to a position as shown in FIG. 9 wherein the inbound and outbound lines feed into the cannula from a substantially vertical direction parallel with the medial plane of the patient.

This adaptability further ensures that any bending of the cannula during reconfiguration between the nasal route and the oral route does not crimp or otherwise impede the transport of the insufflation gas or exhaled air from the patient. Moreover, the configurational adaptability allows gas insufflation line and the exhalation monitoring line to be drawn together in substantially parallel alignment as shown in FIG. 9 so as not to interfere with the surgeon's access to the patients face or head.

Accordingly, the gas insufflation line and the exhalation monitoring line can be positioned cranially/superiorly, or caudally/inferiorly from the mouth, either of which may be a preferred or optimal positioning of the tubing in contrast with the lines extending laterally from each side of the cannula across one or both cheeks of the patient as shown in FIG. 7. In addition, the system can optionally include a sliding retention ring encircling both tubes, and capable of moving the longitudinal axis of the tubes to allow the two tubes to be drawn together or spread apart as needed.

The elongate body portion and lip flange may be formed from a rigid, preferably lightweight material, and can be metal, plastic, or other suitable medical grade material capable of being formed in the configurations described. A variety of suitable medical grade materials for manufacturing medical devices are currently available and known in the art. Most commonly, devices such as the present invention are fabricated from polymeric materials. Such polymers may include but are not limited to thermosetting polymers, thermoplastic polymers, and mixtures thereof. Moreover, it will be further apparent to those skilled in the art that selection of such polymers or copolymers should be such that the resulting polymeric matrix is of sufficient durability and rigidity.

It will be further appreciated that such materials are exemplary only, as other materials may be equally or more suitable. Preferably, the elongate body portion and lip flange are formed by injection-molded plastic or polymeric materials, such as thermoplastic polyethylene-based polymers of suitable medical grade.

Importantly, those skilled in the art will appreciate that the material forming the bite surface and pharyngeal sections of the elongate body will be limited only to the extent that it adequately serves to provide a suitably rigid structure that may be inserted into the intraoral and pharyngeal region and is of sufficient strength to maintain an open airway in the patient and to withstand any forces exerted by the mandibular and maxillary arches while placed in the patient's oral cavity.

As discussed above, the delivery conduits of the present invention may be integrally formed with the bite block elongate body at the time of manufacture or attached by any suitable means such as a medical grade glue or other attachment apparatus. In the former approach, the conduits will be formed from the same polymeric material as the bite block whereas in the second approach, the conduits may be formed by a different type of material and subsequently attached provided that the material is of adequate medical grade and can be adequately formed into a suitable conduit. Preferably, the delivery conduits will be formed from suitable medical grade tubing having suitable dimensions for placement into the bite block channel as discussed above.

As mentioned above, the bite block of the present invention is ideal for use in certain treatments and surgical procedures where gas insufflation and exhalation monitoring by nasal cannulation is not practical and there is no need to accommodate insertion of surgical instruments, endoscopic devices or intubation apparatus. The device is particularly useful in surgical procedures involving facial reconstruction or cosmetic modifications where the patient does not require intubation or intraoral insertion of other medical apparatus such as surgical instruments or endoscopes. Additionally, the present invention may also be used in outpatient applications such as sleep apnea studies.

In particular, the subject invention comprises a method for insufflation and ETCO₂ monitoring of a patient under anesthesia, and particularly under anesthesia for undergoing surgery or a medical procedure involving the head or face, wherein the method comprises providing an oral bite block according to the subject invention, which is capable of engaging, or matingly engages, a nasal insufflation cannula, such that the cannula is useful for both nasal and oral oxygen delivery and ETCO₂ monitoring.

The method can further comprise alternating the position of the cannula, as needed or desired, between nasal and oral positions. For example, the method can comprise positioning the cannula at the nares for nasal insufflation and monitoring as shown in FIG. 7 or, alternatively, engaged with the bite block placed in the mouth of the patient for oral insufflation and monitoring as shown in FIGS. 8 and 9.

For example, nasal insufflation can be useful and advantageous when a procedure is being undertaken to the lower (lip, mouth or chin) area of the face or the upper (forehead or brow) area of the face, whereas oral insufflation can be useful and advantageous when the surgical procedure is undertaken in the middle (nose, cheek, etc.) of the face. When a single procedure may involve procedures done to the upper, middle, and lower areas of the face, alternating the position of the cannula between the nasal area and the oral bite block.

Placement of the device in a patient can be achieved by any number of techniques known in the medical and surgical arts. Importantly, the bite block of the present invention may include a variety of dimensions suitable to accommodate the anatomy of either adults or children based on dimensional considerations well known in the medical and anatomical arts.

Advantageously, the bite block of the present invention does not require an attachment mechanism such as a strap or tie, for example, to retain position of the bite block during use. Accordingly, the bite block of the invention can be a strapless or tie-less bite block. Despite the foregoing, such a securing means can be optionally included if desirable.

The device may be used to deliver insufflation gas through both delivery conduits where exhalation monitoring is not desired. Alternatively, both delivery conduits may be used for exhalation monitoring however such a usage is not likely to be encountered. Most often, one of the delivery conduits will be used to deliver and insufflation gas such as oxygen while the other conduit provides for transport of expired air from the patient to a monitoring device such as an ETCO₂ unit.

While the invention has been described in its preferred forms or embodiments with some degree of particularity, it is understood that the detailed description as set forth herein has been provided only by way of example and that numerous modifications, changes, variations, substitutions and equivalents may be available as well as alternative details regarding construction, fabrication, and use, including the combination and arrangement of parts, all of the foregoing being readily apparent to those skilled in the art without departing from the spirit and scope of the present invention as described and claimed.

1. An intraoral bite block for preventing dental occlusion and providing for oral insufflation of an anesthetized patient using a nasal cannula, said bite block comprising:
an elongate body having a proximal and a distal end, the ends opposite one another relative to the longitudinal axis of the elongate body, wherein the proximal end terminates in at least one lip flange which engages an external aspect of a lip of the patient, the elongate body further comprising an upper and lower bite surface positioned adjacent and distal to the lip flange to mate with the upper and lower dental arches of the patient, respectively, the elongate body having a length sufficient to extend distally into the oropharyngeal cavity of the patient;
at least one, but not more than two, gas conduits extending distally along the longitudinal axis of the elongate body from the lip flange and extending distally beyond the upper and lower bite surface;
each conduit being configured at its proximal end to matingly and detachably couple with an insufflation gas source, exhalation gas monitor, or nasal cannula.

2. The bite block of claim 1 comprising a conduit formed along the longitudinal axis of the elongate body wherein the conduit is sized to receive an oxygen insufflation tube and an exhalation tube.

3. The bite block of claim 2 wherein the conduit is divided by a septum forming first and second conduits.

4. The bite block of claim 1, wherein the first and second conduits form a right and a left channel for disposing and affixing an insufflation tube in one of said right or left channels and disposing and affixing an exhalation tube in the other channel.

5. The bite block of claim 4 wherein the insufflation tube transports oxygen to the patient and the exhalation tube transports exhaled gas from the patient.

6. The bite block of claim 4 wherein the insufflation tube and exhalation tube are permanently affixed within the channels along the longitudinal axis of the elongate body.

7. The bite block of claim 1 wherein the lip flange is integrally formed with the elongate body of the bite block.

8. The bite block of claim 1 wherein the bite surface comprises a groove to receive occlusal surfaces of teeth of the patient.

9. The bite block of claim 1 wherein the bite surface is cushioned.

10. The bite block of claim 1 comprising an upper lip flange extending superiorly and perpendicular to the longitudinal axis of the elongate body and a lower lip flange extending inferiorly and perpendicular to the longitudinal axis of the elongate body.

11. The bite block of claim 1 wherein the elongate body serves as a tongue depressor for maintaining an open oropharyngeal airway in the patient.

12. The bite block of claim 1 wherein the elongate body is curved for anatomical conformity with the oropharyngeal cavity of the patient.

13. The bite block of claim 1, wherein the elongate body has a cross-sectional “I” shape having a longitudinal partitioning septum disposed in a vertical orientation along the longitudinal midplane of the elongate body and substantially planar upper and lower projections formed perpendicular to the septum thereby forming two channels on each side of the septum, wherein a first conduit for gas insufflation is disposed and affixed within one channel and a second conduit for gas exhalation is disposed and affixed within the other channel.

14. The bite block of claim 13 wherein the channels comprise open sides.

15. The bite block of claim 13 wherein the channels are closed, comprising exterior side walls parallel to the longitudinal partitioning septum.

16. The bite block of claim 1 wherein the gas conduit comprises a connector between the proximate end of the conduit and the port of the nasal cannula.

17. The bite block of claim 1 comprising two conduits wherein the proximate ends of the conduits extend out past the lip flange.

18. The bite block of claim 6 wherein the connector extends out past the lip flange.

19. The bite block of claim 16 comprising two gas conduits and a connector is permanently affixed to each conduit.

20. The bite block of claim 1 comprising an inhalation conduit and an exhalation conduit, wherein the gas conduits are spatially configured to matingly couple to inhalation and exhalation ports of a conventional nasal cannula.

21. The bite block of claim 1 wherein the bite block excludes a third conduit for receiving intubation or instrumentation.

22. A method for providing oral insufflation to, and exhalation monitoring of, an anesthetized patient undergoing a procedure requiring access to an area of the face or head, the method comprising:
positioning an intraoral bite block of claim 1 into an oropharyngeal cavity of the anesthetized patient, and
detachably coupling a nasal cannula to the bite block to avoid cannular interference with access to the face or head area undergoing the procedure.

23. The method of claim 22 wherein he intraoral bite block remains in the oropharyngeal cavity when the nasal cannula is detached from the bite block and positioned for nasal insufflation.

24. A system for alternatively providing oral and nasal gas insufflation to, and exhalation monitoring of, an anesthetized patient, the system comprising:
an intraoral bite block comprising an elongate body having a proximal and a distal end, the ends opposite one another relative to the longitudinal axis of the elongate body, wherein the proximal end terminates in at least one lip flange which engages an external aspect of a lip of the patient, the elongate body further comprising an upper and lower bite surface positioned adjacent and distal to the lip flange to mate with the upper and lower dental arches of the patient, respectively, the elongate body having a length sufficient to extend distally into the oropharyngeal cavity of the patient, the elongate body further comprising at least one, but not more than two, gas conduits extending distally along the longitudinal axis of the elongate body from the lip flange and extending distally beyond the upper and lower bite surface with each conduit being configured at its proximal end to matingly and detachably couple with a port of a nasal cannula, and
a nasal cannula for providing gas insufflation and exhalation monitoring of a patient, the cannula being capable of matingly coupling with and rapidly detachable from said bite block, wherein the nasal cannula comprises a partition between an inhalation side an exhalation side of the cannula, said partition being partially divided or split to allow the inhalation and exhalation sides of the cannula to bend up to 90° from its normal position and be configured in substantially parallel alignment with one another.

26. The system of claim 25, wherein the cannula comprises insufflation tubing extending to an insufflation gas source and exhalation tubing extending to a gas monitor, said insufflation tubing and exhalation tubing being drawn together and adjoined in a side-by-side configuration by an adjoining means engaging each of the insufflation tubing and exhalation tubing, the adjoining means being slidingly adjustable along the lengths of said tubing.
